(19) United States
(12) Patent Application Publication
(10) Pub. No.: US 2012/0239070 A1
(43) Pub. Date: Sep. 20, 2012

(54) Cutting needle for urological and other surgical procedures

(76) Inventors: Bandula Wijay, (US); Omar Durrani, Houston, TX (US); Nandhika Wijay, Friendswood, TX (US)

(21) Appl. No.: 13/065,190

(22) Filed: Mar. 15, 2011

(51) Int. Cl. A61B 17/34

(52) U.S. Cl. 606/185

(57) ABSTRACT

A cutting needle assembly for making a percutaneous incision in a living body such as that required for supra-pubic catheterization. The cutting needle of the assembly consists of a hollow beveled tip needle shaft with a surgical blade suitably attached thereto at a distance proximal from the beveled tip, the blade extending radially outward from the needle shaft and extending along a length thereof, for use in creating cuts in tissue adjacent the needle to provide an opening of larger size than the needle puncture for the insertion of other medical instruments including dilators and catheters.

### FIELD OF THE INVENTION

The present invention relates to a cutting needle with an attached cutting blade which can be used in urological procedures, such as supra-pubic catheterizations, or other surgical procedures, and which makes a perforation in the skin and fascia for easier insertion of a catheter or other medical device.

### BACKGROUND OF THE INVENTION

Urinary retention, which can be either acute or chronic, is the inability to voluntarily urinate. Acute urinary retention is a sudden inability to urinate which can result from many different types of causes, and is considered a medical emergency requiring prompt action. With chronic urinary retention, patients are still able to urinate, but have trouble starting a urinary stream or emptying the bladder completely. Although not as urgent as acute urinary retention, patients with chronic urinary retention still require some form of medical treatment in order to avoid developing any serious or long-term complications. Obstructions of the lower urinary tract, also called Bladder Outlet Obstructions (BOO), at or below the neck of the bladder, can cause urinary retention. Bladder outlet obstructions can arise from intrinsic sources, such as bladder stones, urethral strictures, neurological disorders, urinary tract infection, and Benign Prostatic Hyperplasia (BPH), or extrinsic sources, such as cancer, surgical procedures, and encroachment from surrounding tissues.

Acute urinary retention is the most common urologic emergency and most often arises from benign prostatic hyperplasia. BPH, a common affliction in men over 60, is the enlargement of the prostate gland that occurs as a result of hormonal changes as men age. Since the prostate gland surrounds the urethra just below the bladder neck, BHP will typically compress and occlude the urethra, causing acute urinary retention. Acute urinary retention is managed by immediate and complete decompression of the bladder through catheterization. Standard transurethral, or indwelling, catheters, such as the common Foley catheter, have been used in the treatment of urinary retention for many years.

However, if urethral catheterization is unsuccessful or contraindicated, the patient should be referred immediately to a physician trained in advanced catheterization techniques, such as the insertion of a catheter into the bladder supra-pubically.

Supra-pubic catheterization is a routinely used procedure to drain the bladder of urine by passing a catheter percutaneously through the anterior abdominal wall, just above the pubic bone. The supra-pubic catheterization procedure is usually performed in the operating room, under general or local anesthesia, using blind, fluoroscopic-guided, or ultrasound-guided percutaneous trocar puncture. Supra-pubic catheters have become more prevalent than indwelling urethral catheters for patients requiring long-term catheterization, such as patients with neurological disorders (e.g. multiple sclerosis), acute urinary retention, and elderly patients who are unfit for prostate procedures such as Transurethral Resection of the Prostate (TURP). Supra-pubic catheterization is also indicated when transurethral catheterization is contraindicated or technically not possible to relieve urinary retention stemming from urethral injuries, urethral obstruction (strictures), benign prosthetic hyperplasia, and prostate cancer. However, supra-pubic catheterization cannot be used in patients with known or suspected carcinoma of the bladder, with a tendency of hemorrhaging, that have had recent surgery of the lower abdomen, who are pregnant, or when distention of the bladder is not possible. Supra-pubic catheterization is a common procedure (in both elective and emergency situations) which may be undertaken by a range of clinicians in a variety of settings. The procedure is often used during emergency bladder outlet obstructions where urethral catheterization is unsuccessful. Although not considered a high risk procedure, complications can occur, such as peritoneal perforation, hematuria, and infection. Recent supra-pubic catheterization procedures employ the Seldinger technique for catheter insertion. With this technique, a small incision is made in the skin superior to the pubic bone to allow easier insertion of the needle. The bladder is then punctured using an 18-gauge needle. Care must be used so that the needle does not puncture the posterior wall of the bladder or other pelvic structures, including the bowel. After confirming the position of the needle in the bladder by aspiration, a guidewire is introduced through the needle. The needle is then removed, leaving the guidewire in the bladder. A trocar and sheath are then introduced over the guidewire and pushed through the incision until they are safely in position within the bladder. The guidewire and trocar are then removed and a Foley, or Foley-type, catheter is inserted into the bladder for continual drainage and disposal of urine.

The present invention relates to a specialized cutting needle for supra-pubic catheterization procedures, which eliminates the need for a trocar. The cutting needle of the present invention consists of an 18 gauge, hollow, beveled-tip needle with a small surgical blade that is welded to the needle tube approximately 1.7 centimeters above the sharp beveled tip. There are only a few other cutting needles in the prior art. For instance, Lawrence in U.S. Pat. No. 6,048,354 describes a sliding knife and needle assembly for creating a percutaneous incision, or portal, in the body for arthroscopic surgery, endoscopic surgery, or other surgical procedures used to visualize internal anatomical structures. The 18 gauge needle of this assembly is solid, not hollow. Therefore, it is unsuitable for use in supra-pubic catheterization procedures because it cannot facilitate the inclusion of a guidewire. Tal, in U.S. Pat. No. 6,716,228, describes a vascular access device consisting of a sliding blade that is attached to a needle. After the needle is inserted into a vein or artery of the patient, the blade slides along the needle's length and makes an incision in the skin adjacent to the inserted needle. This device reduces the number of surgical instruments required and ensures that the incision is adjacent to the needle for the accurate insertion of guidewires and catheters. However, the blade in this device is not anchored in a fixed position, an aspect that is required for creating a perforation for supra-pubic procedures. In addition, Camrud, in U.S. Pat. No. 6,921,387, describes a beveled-tip vascular needle equipped with a laterally extended blade that is positioned just above the beveled tip. The blade attached to this vascular needle creates an incision in the vessel that reduces the risk of tearing the vessel when a catheter is subsequently inserted into the artery or vein. Since the blade is adjacent to the tip, the cutting needle of this device cannot enter the tissue as the surface at the entry point is too large and as there is little or no anchoring of the needle in the tissue for repeated insertions.

### SUMMARY OF THE INVENTION

The cutting needle assembly of the present invention comprises a stainless steel, hollow needle with a beveled tip and a surgical blade that is suitably attached to the needle, such as by welding, to the at a distance proximally from the beveled tip. The cutting needle assembly of the present invention allows for easier insertion of a supra-pubic catheter, or in similar procedures that require the creation of a percutaneous opening for the insertion of catheters, such as a common Foley catheter; optical devices, and other instruments, into a body cavity. The needle functions as a back support for the cutting operation of the cutting blade, and by acting as a guide, allows for a more precise cutting direction. The blade is positioned proximally from the beveled tip so that the user can puncture the skin with the needle before cutting, thereby providing an anchoring means for the needle in order for the blade to incise the tissue at different angles from the same puncture site. The blade is curved in order to allow for a smooth cutting action as it slides through the tissue. The cutting needle of the present invention creates a large hole for easier insertion of indwelling catheters or other devices, and eliminates the use of a trocar, an instrument which requires a significant amount of force, produces a large puncture hole, and which can perforate the bowel or other organs. As a result, the present invention makes a puncture in the skin, and underlying fascia, that is easier to produce, and which reduces both the trauma and the chance of injury to the patient.

### BRIEF DESCRIPTION OF THE DRAWINGS

FIG. 1. is a perspective view of the preferred embodiment of the cutting needle of the present invention.

### DETAILED DESCRIPTION OF THE PREFERRED EMBODIMENT

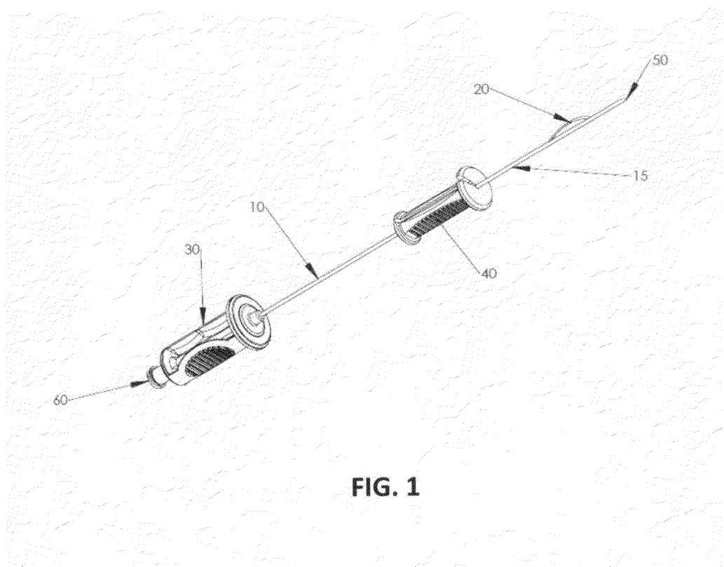

FIG. 1 shows the preferred embodiment of the cutting needle assembly (10) made in accordance with the teachings of the present invention. The cutting needle assembly (10) is preferably composed of a hollow, 18 gauge, stainless steel needle shaft (15), with a beveled tip (50) and a stainless steel cutting blade (20) that is attached to the needle shaft (15), such as by welding, and extends radially outwardly therefrom, along a length of the needle shaft (15), the cutting blade (20) being curved to provide a smooth sliding motion as it incises through tissue surrounding a puncture created with the needle assembly (10). The cutting blade (20), in a preferred embodiment, is welded to the needle shaft (15) approximately 1.7 centimeters above the sharp beveled tip (50) and is approximately 20-25 mm in length. The cutting needle assembly (10) is equipped with a hub (30) at an end opposite that of the beveled tip (50) and a gripper (40). The hub (30) is attached to the end of the needle shaft (15) preferably by gluing or insert molding. The gripper (40) is removably engaged over the needle shaft (15), is slidable therealong, is made of a compressible material, and is used as a means for holding the needle (10) as it is inserted into the patient, creating a puncture, and as a means for reorienting the blade (20) as necessary. The hub (30) has a throughbore (60) therein which aligns with the hollow interior of the needle shaft (15) so that the cutting needle (10) can be passed over a guidewire. After the initial insertion into the patient, the cutting needle (10) is partially withdrawn from the initial puncture just until the blade (20) has exited the skin, leaving the beveled tip (50) within the original puncture, is rotated 90 degrees, and reinserted into the patient. The position of the blade (20), proximal to the beveled tip (50), allows the beveled tip (50) of the needle (10) to create an anchor for the cutting blade (20) during the process of cutting through tissue surrounding the puncture made with the needle assembly (10). This process is repeated until the desired number of cuts through the skin and fascia about the puncture are created with the blade (20).

We claim:

1. A cutting needle assembly comprising:
a needle comprising a hollow cylindrical shaft having a beveled tip at one end;
a cutting blade suitably attached to the needle proximally from the beveled tip and extending radially outwardly of the shaft and along a length thereof; and
a hub at a proximal end of the shaft with a throughbore therein aligned with an interior of the hollow shaft for the insertion of a guidewire therethrough.

2. The cutting needle assembly of claim 1 wherein:
a releasably engaged compressible gripper for assisting with the insertion of the cutting needle assembly into and through tissue is slidably provided along the length of the needle.

3. The cutting needle assembly of claim 1 wherein:
the cutting blade is curved to provide a smooth sliding motion as it incises through tissue.

4. The cutting needle assembly of claim 1 wherein:
the needle shaft is composed of steel.

5. The cutting needle assembly of claim 1 wherein:
the position of the blade, proximal to the beveled tip, allows the tip of the needle to create an anchor for the needle assembly during the cutting of the tissue surrounding the needle.

6. The cutting needle assembly of claim 5 wherein:
the cutting blade is positioned approximately 1.7 centimeters above the sharp beveled tip.

7. The cutting needle assembly of claim 1 wherein:
the cutting blade is composed of stainless steel.

8. The cutting needle assembly of claim 7 wherein:
the cutting blade is approximately 20-25 mm in length.
